(19) United States
(12) Patent Application Publication
(10) Pub. No.: US 2025/0312411 A1
(43) Pub. Date: Oct. 9, 2025

(54) Treatment of Multi-Drug-Resistant Cancers Using Nanoparticulate Drug Delivery Vehicles

(71) Applicant: Northeastern University, Boston, MA (US)

(72) Inventors: Mansoor AMIJI, Attleboro, MA (US); Lara MILANE, Boston, MA (US)

(21) Appl. No.: 19/170,656

(22) Filed: Apr. 4, 2025

Related U.S. Application Data
(60) Provisional application No. 63/574,879, filed on Apr. 4, 2024.

(51) Int. Cl. A61K 38/10; A61K 9/1272; A61K 9/51; A61K 31/155; A61K 31/407; A61K 38/17

(52) U.S. Cl. CPC A61K 38/10; A61K 9/1272; A61K 9/5146; A61K 9/5153; A61K 31/155; A61K 31/407; A61K 38/17

(57) ABSTRACT

Pharmaceutical compositions and methods provide treatment of solid tumors based on simultaneously preventing the development and spread of multi-drug resistance (MDR) and a metastatic phenotype. This technology operates through the inhibition of two types of intercellular communication within the tumor microenvironment—tunneling nanotubes and extracellular vesicles, both of which promote the spread of MDR. Nanoparticle delivery of both an inhibitor of tunnelling nanotubes and an inhibitor of extracellular vesicle release work synergistically to trap and improve the effectiveness of anticancer drugs in cells of the tumor and permit the use of lower doses of anticancer drugs, with reduction in harmful side effects.

### CROSS-REFERENCE TO RELATED APPLICATIONS

This application claims priority to U.S. Provisional Application No. 63/574,879, filed on 4 Apr. 2024, which is hereby incorporated by reference in its entirety.

### BACKGROUND

A subset of cancer cells in solid tumors are multi-drug resistant (MDR). MDR often spreads throughout the tumor microenvironment (TME) and drug sensitive cells are converted into MDR cells⁷¹⁻⁸⁰. Intercellular communication via tunnelling nanotubes (TNTs) and extracellular vesicles (EVs) within the TME drives the progression and spread of MDR and metastasis⁸⁻¹⁴. The direct transfer of cellular content from the cancer cells to the neighboring cells in the tumor microenvironment, such as anticancer drugs, via EVs and TNTs is an important mechanism which ensures cellular survival and avoids cell death resulting from anti-tumor drug retention^{8,9,15,16}. Drug transfer to cells without anticancer drug overload helps these cells to adapt to the drug's mechanism and aids in the development of drug resistance^{8,9,17}. Mitochondria have been well-characterized as being transferred by TNTs in tumor microenvironments^{15,16,18-26}. The TNT transfer of mitochondria from non-cancerous cells such as macrophages, epithelial cells, and stromal cells to cancer cells promotes cancer cell survival, whereas TNT mitochondrial transfer from cancerous cells to non-cancerous cells is associated with either malignant transformation of the cell or immune function depletion^{15,16,18-26}. Intercellular exchange of microRNAs via TNTs and EVs also propagates the MDR cellular phenotype within the TME²⁷. TNT communication between tumor cells and tumor associated macrophages also propels tumor aggressiveness and metastasis⁹. F-actin is the primary structural component of TNTs, while mitochondria are (1) required for active TNT synthesis and (2) are actively exchanged between cells via established TNTs^{8,9,15,16}.

EVs are released when multi-vesicular bodies (MVBs) containing EVs fuse with the plasma membrane via soluble N-ethylmaleimide-sensitive factor attachment protein receptors (SNARE proteins)^{11,28}. EVs are critical to the initiation of metastasis, as EVs containing matrix metalloproteinases (MMPs) are released from the leading edge of a cell before cell movement occurs¹¹⁻¹³. EV's MMPs digest the extracellular matrix and thus pave the way for the metastasizing cell to begin invasion¹¹⁻¹³. EVs also establish pre-metastatic niches, arriving at a metastatic site and priming the site for recruitment of circulating tumor cells^{12,14}.

While intercellular communication via tunneling nanotubes (TNTs) and extracellular vesicles (EVs) within the tumor microenvironment (TME) is critical to the development and spread of multidrug resistance (MDR) and metastasis of malignant cells, there are currently no cancer therapies that simultaneously target these intercellular communication processes.

### SUMMARY

The present technology provides a novel and unique treatment for solid tumors, and is in particular aimed at preventing the development and spread of multi-drug resistance phenotypes and metastasis. Both multi-drug resistance (MDR) and metastasis are more often associated with recurrent disease and poor prognosis. The technology operates through the inhibition of two types of intercellular communication within the tumor microenvironment (TME)—tunneling nanotubes and extracellular vesicles, both of which promote the spread of MDR. Since the use of the present technology traps anticancer drugs in cells of the TME, it increases the efficacy of anticancer drug treatment and permits the use of lower doses of anticancer drugs, with reduction in harmful side effects.

Thus, by blocking mechanisms of intercellular communication via tunneling nanotubes (TNTs) and extracellular vesicles (EVs) within the tumor microenvironment (TME), the present compositions and methods inhibit the development and spread of MDR and metastasis, and also enable reduction of the dose of antitumor drugs.

The pharmaceutical compositions of the present technology combine two active agents: an F-actin inhibitor that prevents TNT formation and an anti-SNARE (soluble N-ethylmaleimide-sensitive factor attachment protein receptor) peptide that inhibits EV release. The two active agents can be packaged inside of the same delivery vehicles, or in separate delivery vehicles that can be mixed, or co-administered, or sequentially administered, or administered together according to a treatment plan, for synergistic effect. F-actin is the cytoskeletal component of TNTs, and preventing its polymerization prevents the formation of TNTs or leads to their degradation. SNARE proteins mediate both the fusion of TNTs with neighboring cells and the release of EVs into the extracellular space, where they can be taken up by neighboring cells. The SNARE-mediated release of EVs can be inhibited by anti-SNARE peptides, for example.

One aspect of the technology is a pharmaceutical composition for treating a solid tumor in a mammalian subject. The pharmaceutical composition includes a plurality of nanoparticles that contain an inhibitor of extracellular vesicle release and/or an inhibitor of tunneling nanotube formation. Preferably, the nanoparticles contain both types of inhibitor. The nanoparticles can be, for example, cationic liposomes or biodegradable polymeric nanoparticles which encapsulate the inhibitors, and preferably are capable of uptake by mammalian cells, such as tumor cells. For example, the cationic liposomes or biodegradable polymeric nanoparticles can be taken up by mammalian cells via an endosomal mechanism, resulting in release of the inhibitors into the cytoplasm of the cell.

Another aspect of the technology is a method of treating a solid tumor-forming cancer in a mammalian subject, such as a human subject. The method includes providing a pharmaceutical composition such as described above and administering the pharmaceutical composition to the mammalian subject, whereby metastasis of malignant cells out of a tumor of the mammalian subject is reduced or avoided as a result of administering the pharmaceutical composition, and/or whereby the development and/or spread of multi-drug resistance by tumor cells within a solid tumor of the subject or within the subject's body is inhibited.

Yet another aspect of the technology is a pharmaceutical kit, that includes (i) a first pharmaceutical composition containing a plurality of first nanoparticles comprising an inhibitor of extracellular vesicle release, and (ii) a second pharmaceutical composition containing a plurality of second nanoparticles comprising an inhibitor of tunneling nanotube formation.

The technology can be further summarized in the following listing of features.
1. A pharmaceutical composition for treating a solid tumor in a mammalian subject, the pharmaceutical composition comprising a plurality of nanoparticles comprising an inhibitor of extracellular vesicle release and/or an inhibitor of tunneling nanotube formation.
2. The pharmaceutical composition of feature 1, wherein the inhibitor of extracellular vesicle release is an anti-SNARE agent.
3. The pharmaceutical composition of feature 2, wherein the anti-SNARE compound is a peptide comprising the amino acid sequence SAAEAFAKLYAEAFAKG (SEQ ID NO:1) or a fragment or variant thereof having at least 70%, at least 80%, at least 90%, at least 95%, at least 98%, or at least 99% identity to SEQ ID NO:1.
4. The pharmaceutical composition of feature 2, wherein the anti-SNARE compound is a peptide comprising the amino acid sequence EEVLQLMRRTSEL (SEQ ID NO:2), NRRLQQTQAQVDEVVDIMRVNVDKVLER (SEQ ID NO:3), or DQKLSELDDRADALQAGASQFETSAAKLKRKYWWKN (SEQ ID NO:4), or a fragment or variant thereof having at least 70%, at least 80%, at least 90%, at least 95%, at least 98%, or at least 99% identity to any of SEQ ID NOS: 2-4.
5. The pharmaceutical composition of feature 3 or feature 4, wherein the anti-SNARE compound is a variant of any of SEQ ID NOS: 1-4 comprising from 1 to 10, from 1 to 5, or from 1 to 3 conservative amino acid substitutions.
6. The pharmaceutical composition of any of the preceding features, wherein the inhibitor of tunneling nanotube formation is an inhibitor of F-actin.
7. The pharmaceutical composition of any of the preceding features, wherein the inhibitor of tunneling nanotube formation is chaetoglobosin A, cytochalasin B, or metformin.
8. The pharmaceutical composition of any of the preceding features, wherein the nanoparticles comprise cationic liposomes or biodegradable polymeric nanoparticles, and wherein the cationic liposomes or polymeric nanoparticles encapsulate the inhibitor of extracellular vesicle release and/or the inhibitor of tunneling nanotube formation.
9. The pharmaceutical composition of feature 8, wherein the nanoparticles are cationic liposomes.
10. The pharmaceutical composition of feature 6, wherein the cationic liposomes comprise 1,2-dioleoyl-3-trimethylammonium-propane (DOTAP) as the cationic lipid and further comprise one or more components selected from the group consisting of a neutral phospholipid, cholesterol, and a hydrophilic polymer such as polyethylene glycol (PEG).
11. The pharmaceutical composition of feature 10, wherein the cationic liposomes have a zeta potential greater than about +30 mV or less than about −30 mV.
12 The pharmaceutical composition of feature 8, wherein the nanoparticles are biodegradable polymeric nanoparticles
13. The pharmaceutical composition of feature 12, wherein the polymeric nanoparticles comprise one or more biodegradable polymers selected from the group consisting of gelatin, chitosan, cellulose, cellulose derivatives, poly(ε-caprolactone) (PCL), polyethylene glycol (PEG), poly(lactic-co-glycolic acid) (PLGA), polylactic acid (PLA), polyglycolide, PEG-PLA diblock copolymer, PEG-PLGA diblock copolymer, PEG-PCL diblock copolymer, PCL-b-PEG-b-PCL co-polymer, polypropylene glycol (PPG), polyacrylic acid, polyacrylamide, poly(N-isopropylacrylamide), polyethylene oxide-polypropylene oxide-polyethylene oxide (PEO-PPO-PEO) triblock copolymer, hyaluronic acid, polyethylene oxide, polypropylene oxide, alginic acid, poly(beta-aminoester) (PBAE), poly(glycolic acid) (PGA), and alginate.
14. The pharmaceutical composition of any of the preceding features, wherein the anti-SNARE compound and the inhibitor of tunneling nanotube formation are co-localized within the same cationic liposomes or within the same biodegradable polymeric nanoparticles.
15. The pharmaceutical composition of any of features 1-13, wherein the anti-SNARE compound and the inhibitor of tunneling nanotube formation are localized within separate cationic liposomes or within separate biodegradable polymeric nanoparticles.
16. A method of treating a solid tumor-forming cancer in a mammalian subject, the method comprising administering the pharmaceutical composition of any of the preceding features to the mammalian subject.
17. The method of feature 16, wherein the solid tumor-forming cancer is selected from the group consisting of breast cancer, endometrial cancer, ovarian cancer, pancreatic cancer, prostate cancer, lung cancer, brain cancer, liver cancer, colorectal cancer, stomach cancer, esophageal cancer, skin cancer, and head and neck cancer.
18. The method of feature 16 or feature 17, wherein release of extracellular vesicles and/or formation, number, or density of tunneling nanotubes are inhibited in a solid tumor of the mammalian subject.
19. The method of any of features 16-18, wherein metastasis of cancer cells out of the tumor and/or development or spread of multidrug resistant cancer cells within the tumor or within the subject are inhibited.
20. The method of any of features 16-19, further comprising administering an anticancer drug to the mammalian subject, prior to, concomitantly with, or after administration of said pharmaceutical composition.
21. The method of feature 20, wherein the anticancer drug is administered at a lower dose than would be administered if the pharmaceutical composition of any of features 1-15 were not also being administered.
22. The method of feature 21, wherein toxicity or another side effect of the anticancer drug is reduced due to its lower dose.
23. A pharmaceutical kit, comprising;
   (i) a first pharmaceutical composition comprising a plurality of first nanoparticles comprising an inhibitor of extracellular vesicle release; and
   (ii) a second pharmaceutical composition comprising a plurality of second nanoparticles comprising an inhibitor of tunneling nanotube formation.
24. The pharmaceutical kit of feature 23, wherein the plurality of first nanoparticles comprise cationic liposomes or biodegradable nanoparticles that encapsulate the inhibitor of extracellular vesicle release; and wherein the plurality of second nanoparticles comprise cationic liposomes or biodegradable nanoparticles that encapsulate the inhibitor of tunneling nanotube formation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show characterization of tunnelling nanotubes (TNTs) in drug sensitive and multidrug resistant triple negative breast cancer cells (MDA-MB-231), endometrial cancer cells (HEC1B), and uterine serous carcinoma cells (USC; Ark1). F-Actin and mitochondria were fluorescently labeled and quantified using 3D fluorescence analysis software.

FIG. 2 shows the ratios of mitochondria to F-actin in TNTs for the indicated cells and growth conditions. The results indicate that TNTs in MDR cells are more actively transporting mitochondria, or there are more newly synthesized TNTs, or both phenomena are occurring simultaneously. F-Actin and mitochondria were fluorescently labeled and quantified using 3D fluorescence analysis software.

FIGS. 3A-3D show brightfield and fluorescence microscopy studies of untreated drug sensitive and MDR cells. Cells were seeded at 10,000 cells per 35 mm IBIDI glass bottom dish and grown under normoxic conditions (drug sensitive cells) or hypoxic conditions (MDR cells grown at 0.5% O₂) for 5 days. Cells were stained with 62.5 nM SiR-Actin dye (red: F-actin) for 60 min and with 62.5 nM MITOTRACKER Green FM for 15 min. Fluorescence microscopy was performed using the 60X objective of a Keyence BZ-X710 microscope. Images were processed using an adaption of mitochondrial network analysis (MiNA) software for FiJi (NIH) to quantify the 3D fluorescent imprint of F-actin and mitochondria in the entire field of view and in manually identified TNTs. For each figure subpart, panel (A) is brightfield, panel (B) is the microscope overlay of all channels, panel C is the merged overlay of all channels after enhancing local contrast, panels D and H are the mitochondrial green channel and F-actin red channel respectively, panels E and I are the binary conversions of D and H, panels F and J are the 2D analysis of the fluorescent imprint in each channel, and panels G and K are the 3D quantification of fluorescence in each channel. These images demonstrate processing for the entire field of view. The same processing was performed on each individual TNT in the image. This is a representative image of 25 separate fields of view. The data from G and K of the total 25 image sets and the individual TNTs were used to generate the graphs in FIGS. 1A-1D and 2. The scale bars on panels G and K are 64 μm.

FIGS. 4A-4C show that MDR cells have increased TNT formation, and that chaetoglobosin A (CA) and metformin each can decrease TNTs.

FIGS. 5A-5B show that the MDR state is associated with increased extracellular vesicle (EV) release, and that anti-SNARE nanoparticles decreased EVs in MDR cells. FIG. 5A shows that the growth rates of drug sensitive and MDR triple negative breast cancer cells (TNBC) were comparable. In FIG. 5B, the numbers of EVs per 1 million cells (FIG. 5B) are shown for administration of blank lipid nanoparticles (BL-LNP) compared to anti-SNARE peptide-containing nanoparticles (AS-LNP).

FIGS. 6A-6C show that anti-SNARE nanoparticles decrease cell migration in drug sensitive (DS) and MDR cells in a wound healing assay. 10 UM and 30 UM anti-SNARE peptide-containing lipid nanoparticles (AS-LNP) reduced cell migration in DS TNBC cells (FIG. 6A) and MDR TNBC cells (FIG. 6B). FIG. 6C shows the results presented in FIGS. 6A and 6B graphed together.

### DETAILED DESCRIPTION

The present technology provides compositions, methods, and kits for treatment of solid tumors, and to inhibit the development of multi-drug resistance and the metastasis of tumor cells. The compositions of the present technology contain a new combination of active agents, each of which affects tumor cell communication via a different mechanism. A first agent prevents the spread of multidrug resistance (MDR) through suppression of tunneling nanotubes (TNT), which promote the MDR phenotype by enabling exchange of mitochondria, anti-tumor drugs, and other factors. A second agent prevents metastasis by inhibiting the release of extracellular vesicles (EVs), which promote transfer of drugs and biomolecules between tumor cells, thereby promoting MDR development. Further, extracellular vesicles from tumor cells can promote the development of metastases at other locations. Both agents can enhance the effectiveness of other anti-tumor agents by causing them to be retained in tumor cells, thus reducing the dose of the other anti-tumor agents for effectiveness.

The first active agent can be any chemical or biological substance that prevents TNT formation and/or promotes the degradation and resorption of TNTs. In a preferred embodiment, the anti-TNT agent is an F-actin inhibitor. F-actin is an important constituent of TNTs, and data presented herein indicate that inhibition of F-actin formation or destabilization of F-actin filaments reduces the number of observable TNTs between tumor cells, particularly TNTs associated with cells exhibiting the MDR phenotype. Examples of suitable F-actin inhibitors are chaetoglobosin A and cytochalasin B. Derivatives of chaetoglobosin A, including MBJ-0038, MBJ-0039, MBJ-0040⁸¹, also can be used. An example of an F-actin independent inhibitor of TNTs is metformin.

The second active agent can be any chemical or biological substance that inhibits EV release from cells of the tumor. Preferred embodiments utilize one or more anti-SNARE peptides, which inhibit the release of membrane-bound vesicles into the extracellular space. Such anti-SNARE peptides also prevent the fusion of TNTs with neighboring cells, and thereby exert a synergistic effect. A preferred anti-SNARE peptide is SAAEAFAKLYAEAFAKG (SEQ ID NO:1). Another preferred anti-SNARE peptide is EEVLQLMRRTSEL (SEQ ID NO:2). Also preferred are fragments or variants of SEQ ID NO:1 or SEQ ID NO:2, such as: (i) fragments or variants of SEQ ID NO: 1 or SEQ ID NO:2 having at least 70%, at least 80%, at least 90%, at least 95%, at least 98%, or at least 99% identity to SEQ ID NO:1 or SEQ ID NO:2; fragments of SEQ ID NO: 1 or SEQ ID NO:2 containing at least 5, at least 6, at least 7, at least 8, at least 9, at least 10, at least 11, at least 12, at least 13, at least 14, or at least 15 consecutive amino acids of SEQ ID NO:1 or SEQ ID NO:2; and variants of SEQ ID NO: 1 or SEQ ID NO:2 containing from 1 to 10, from 1 to 5, or from 1 to 3 conservative amino acid substitutions compared to either SEQ ID NO:1 or SEQ ID NO:2. More preferred are anti-SNARE peptides that inhibit release of EVs from a tumor cell. In order to determine whether a variant or fragment of an anti-SNARE peptide has activity to inhibit EV release, the following assay can be used. EVs can be isolated from an extracellular fluid or culture medium by ultrafiltration followed by size-exclusion chromatography (UF-SEC); EVs then can be quantified using a double-sandwich ELISA assay to measure the expression level of CD63, a highly characteristic tetraspanin found on the extracellular vesicle membrane. Conservative amino acid substitutions in a peptide or polypeptide are substitutions of an amino acid with an equivalent amino acid which do not substantially alter the structure and/or functionality of the peptide or polypeptide. Equivalent amino acids have side chains with similar properties such as bulkiness, polarity (polar or non-polar), hydrophobicity (hydrophobic or hydrophilic), pH-dependent protonatable groups (acidic, neutral or basic), and organization of carbon molecules (aromatic or aliphatic).

Amino acids can be divided into the following classes, within which amino acids are considered equivalent. Substitutions made within the same class are considered conservative substitutions.
1. Amino acids having polar side chains (Asp, Glu, Lys, Arg, His, Asn, Gln, Ser, Thr, Tyr, and Cys)
2. Amino acids having non-polar side chains (Gly, Ala, Val, Leu, lle, Phe, Trp, Pro, and Met)
3. Amino acids having aliphatic side chains (Gly, Ala Val, Leu, lle)
1. Amino acids having cyclic side chains (Phe, Tyr, Trp, His, Pro)
2. Amino acids having aromatic side chains (Phe, Tyr, Trp)
3. Amino acids having acidic side chains (Asp, Glu)
4. Amino acids having basic side chains (Lys, Arg, His)
5. Amino acids having amide side chains (Asn, Gln)
6. Amino acids having hydroxy side chains (Ser, Thr)
7. Amino acids having sulfur-containing side chains (Cys, Met),
8. Neutral, weakly hydrophobic amino acids (Pro, Ala, Gly, Ser, Thr)
9. Hydrophilic, acidic amino acids (Gln, Asn, Glu, Asp), and
10. Hydrophobic amino acids (Leu, lle, Val)

An active agent can be administered, for example as packaged in lipid- or polymer-based nanoparticles, in its active, mature form or in a precursor form that is released in the subject's body or in the target cell to provide the active form.

Compositions of the present technology can be pharmaceutical compositions, comprising one or more active agents and one or more excipients of any kind. Excipients can include, for example, diluents, buffers, carriers, adjuvants, salts, and the like. While pharmaceutical compositions can take many forms, such as liquids, suspensions, solids, tablets, powders, capsules, or aerosols, a preferred form is an aqueous suspension of nanoparticles formulated for parenteral administration, such as intravenous or subcutaneous injection, or direct injection into a tumor or another tissue. Also contemplated are solid forms that release nanoparticles of the present technology, such as over a period of time, into surrounding tissue, or into the circulatory system, the lymphatic system, or the central nervous system. Nanoparticles of the present technology, optionally embedded in a biodegradable polymer matrix, can be implanted anywhere in the subject's body for subsequent release. Implants can be used as part of or in conjunction with a medical device, including an implantable medical device. U.S. Pat. No. 12,121,608 (hereby incorporated by reference in its entirety) describes an implant for delivery through the olfactory epithelium into the brain, bypassing the blood-brain barrier; such an implant can be used to deliver nanoparticles of the present technology to the brain or other parts of the central nervous system. Certain pH-sensitive biodegradable polymers, such as Eudragit (a copolymer of methyl methacrylate and/or other alkyl methacrylates), also can be used for oral delivery of any of the active agents described herein; see, for example, U.S. Pat. No. 11,491,114, which is hereby incorporated by reference in its entirety.

Pharmaceutical compositions for use in the present technology include a plurality of nanoparticles that contain at least two active agents: an inhibitor of extracellular vesicle release and an inhibitor of tunneling nanotube formation. In a preferred embodiment, the nanoparticles contain both types of active agent; more preferably each nanoparticle contains both types of active agent. Also contemplated are pharmaceutical compositions containing a mixture of two or more different populations of nanoparticles, each population containing one or more active agents that are different from the active agent(s) contained in other populations of nanoparticles. Preferably, the nanoparticles are suspended in an aqueous medium, which is preferably isotonic and suitable for injection into the subject's body. In alternative embodiments, the nanoparticles can be suspended in a solid, gel, or other formulation for administration to the subject by implantation, oral delivery, transmucosal delivery, or topical application.

The nanoparticles can be lipid nanoparticles, such as in the form of a suspension of liposomes or micelles that contain, encapsulate, or bind one or more active agents of the present technology. The lipid nanoparticles can be, for example, cationic liposomes that encapsulate one or more active agents within their aqueous lumen, solubilized within the aqueous medium, or bound non-covalently or covalently to the lipid bilayer membrane at its inner or outer surface or within its hydrophobic core. Liposomes can be used in many different formulations, containing different types of phospholipids, sterols such as cholesterol, sphingolipids, and glycolipids, as are well known in the field. Cationic liposomes generally contain one or more species of lipids that are positively charged at the pH range encountered in the body, such as the pH of the blood, intercellular medium, tumor microenvironment, or cytoplasm of tumor cells. One such cationic lipid is 1,2-dioleoyl-3-trimethylammonium-propane (DOTAP). The relative content of cationic, neutral, and/or anionic lipids in liposomes can be adjusted according to need, and will contribute to determining the zeta potential of the liposomes, which can affect their binding and uptake by cells. In preferred embodiments, the zeta potential of the liposomes is greater than about +30 mV or less than about −30 mV. In alternative embodiments, the zeta potential of the liposomes is in a range from about 0 mV to about +100 mV, or from about 0 mV to about −100 mV, or from about +30 mV to about +100 mV, or from about −30 mV to about −100 mV, or from about +40 mV to about +60 mV, or from about −40 mV to about −60 mV. Preferably, the zeta potential is in a range that promotes stability and avoids aggregation of the liposomes. The size of a population of liposomes also can be varied by their method of production, and can affect liposome stability and delivery. The mean vesicle size (diameter) and size distribution of a population of liposomes can be determined, for example, by dynamic light scattering, or by electron microscopy, or using a trapped fluorescent dye, all by known methods. In embodiments, the average (mean) diameter of a population of liposomes suitable for use in the present technology can be, for example, in a range from about 50 nm, about 60 nm, about 70 nm, about 80 nm, about 90 nm, about 100 nm, about 120 nm, about 150 nm, or about 200 nm, as the lower end of the range, to about 80 nm, about 100 nm, about 150 nm, about 200 nm, about 250 nm, about 300 nm, about 350 nm, about 400 nm, or about 500 nm as the upper end of the range, with the proviso that the lower end of the range is smaller than the upper end of the range. Liposomes can be unilamellar or multilamellar. Any known method of forming liposomes and entrapping or binding active agents, and removing unincorporated active agents, can be used to prepare the liposomes. Examples of liposome formation methods include forming a dry lipid film on a surface by solvent evaporation followed by mechanical disruption of the film and sonication, freezing and thawing a lipid suspension, solvent evaporation or dilution, and detergent solubilization of lipids followed by detergent removal. Removal of unincorporated active agents or other substances can be performed, for example, by size exclusion chromatography, filtration, dialysis, or centrifugation.

Nanoparticles can be in the form of biodegradable polymeric nanoparticles containing one or more active agents. Preferably the active agents are non-covalently associated with one or more biodegradable polymers. The biodegradable polymeric nanoparticles can be core-shell nanoparticles, wherein the active agents are incorporated within the core of the particle and are surrounded by a shell of polymer molecules. Alternatively, the active agents can be entrapped within a matrix of polymer molecules. Suitable biodegradable polymers include gelatin, chitosan, cellulose, cellulose derivatives, poly(¿-caprolactone) (PCL), polyethylene glycol (PEG), poly(lactic-co-glycolic acid) (PLGA), polylactic acid (PLA), polyglycolide, PEG-PLA diblock copolymer, PEG-PLGA diblock copolymer, PEG-PCL diblock copolymer, PCL-b-PEG-b-PCL co-polymer, polypropylene glycol (PPG), polyacrylic acid, polyacrylamide, poly(N-isopropylacrylamide), polyethylene oxide-polypropylene oxide-polyethylene oxide (PEO-PPO-PEO) triblock copolymer, hyaluronic acid, polyethylene oxide, polypropylene oxide, alginic acid, poly(beta-aminoester) (PBAE), poly(glycolic acid) (PGA), alginate, and any combinations thereof. Any known method can be used to prepare polymeric nanoparticles containing the active agents, including for example, polymer precipitation by solvent exchange or pH shift or polymerization from precursors. Removal of unincorporated active agents or other substances can be performed, for example, by size exclusion chromatography, filtration, dialysis, or centrifugation. In embodiments, the average (mean) diameter of a population of polymeric nanoparticles suitable for use in the present technology can be, for example, in a range from about 50 nm, about 60 nm, about 70 nm, about 80 nm, about 90 nm, about 100 nm, about 120 nm, about 150 nm, or about 200 nm, as the lower end of the range, to about 80 nm, about 100 nm, about 150 nm, about 200 nm, about 250 nm, about 300 nm, about 350 nm, about 400 nm, or about 500 nm as the upper end of the range, with the proviso that the lower end of the range is smaller than the upper end of the range.

While nanoparticles tend to accumulate in solid tumors due to the enhanced permeability and retention (EPR) effect, which for many applications may be selective enough for treatment, both lipid-based and polymer-based nanoparticles of the present technology can optionally include one or more targeting agents or targeting mechanisms, which promote the selective binding to and uptake by certain target cells, such as tumor cells or the tumor microenvironment of a solid tumor, for example. A listing of antibodies targeting specific tumor types is found at www.cancer.gov/about-cancer/treatment/types/targeted-therapies/approved-drug-list. Any known targeting agent or targeting mechanism can be used, or combinations of two or more targeting agents. Examples include attaching, covalently or non-covalently, antibodies (especially monoclonal antibodies), aptamers, receptor proteins, or receptor ligands to the outer surface of the nanoparticles, wherein the antibodies, aptamers, receptor proteins, or receptor ligands have a binding specificity for cell surface molecules of target cells, such as tumor antigens, receptors, or receptor ligands. Targeting mechanisms also can include surface charge of nanoparticles or the presence of sugars or peptides on the nanoparticle surface. Preferably the nanoparticles remain intact until taken up by target cells, such as uptake into the endosomal compartment of target cells, where the nanoparticles are permeabilized or degraded and release their active agents into the cell. Certain types of polymers can be directed to the endosomal compartment and released into target cells by including oligopeptides as part of the polymer molecule, such as by the inclusion of basic amino acids in oligopeptides added at the ends of polymer molecules; see published patent application US 2018/0250410 A1, which is hereby incorporated by reference in its entirety.

The present pharmaceutical compositions can be used in methods of treating a solid tumor-forming cancer in a mammalian subject, such as a human subject. The methods include providing a pharmaceutical composition such as described above and administering the pharmaceutical composition to the mammalian subject. As a result, development of a multi-drug resistance phenotype by the tumor cells is inhibited, as is the metastasis of malignant cells out of a tumor. The pharmaceutical compositions can be administered by any known method consistent with maintaining their structure and function, and allowing the nanoparticles contained therein to reach their target cells. Administration can be, for example, parenteral, by intravenous or subcutaneous injection, by surgical implantation of a sustained release formulation, oral (with suitable polymeric nanoparticles capable of protecting the active agents), or by topical administration. The subject can be any mammalian subject, and is preferably a human subject. Preferably, the subject is known or suspected to have a solid tumor or a solid-tumor causing form of cancer, at any stage. Successful treatment or prevention can result in, for example, stabilization of the disease, reduction to any degree of tumor size, weight, density, number, or degree of malignancy, increase to any degree of survival or survival expectancy, or reduction to any degree and at any time, of the formation of new tumors or the growth or any tumor existing prior to treatment or prevention protocol.

In addition to administering to a subject a single pharmaceutical composition whose nanoparticles contain both types of active agent (i.e., one directed at inhibiting TNTs and the other inhibiting EV release), the present pharmaceutical compositions can be administered to the same subject in various combinations of two or more separate pharmaceutical compositions, each comprising a different active agent, or a different dose of active agent, or a different type of nanoparticle delivery vehicle (e.g., one or more cationic liposomal nanoparticles plus one or more biodegradable polymeric nanoparticles). The two or more separate pharmaceutical compositions can be administered concurrently or at different times, in the same formulation or in different formulations, and via the same route of administration or different routes of administration.

The present pharmaceutical compositions can be administered as an adjunct therapy to many other types of anti-tumor therapies. In general, any anti-tumor therapy that is directed at inhibiting the growth of cells in a solid tumor, or preventing the development of MDR, or preventing the metastatic spread of cancer cells from a tumor, can benefit from performing the present methods of treatment either before, concurrent with, or after the anti-tumor therapy; preferably the present methods are administered after an anti-tumor therapy. Without intending to limit the invention as an adjunct to anti-tumor therapy, suitable anti-tumor therapies include chemotherapy, radiation therapy, antibody therapy, bispecific antibody therapy, checkpoint inhibitors, small molecule drug therapy, therapeutic cancer vaccines, dendritic cell vaccines, use of natural killer cells, CAR-T cell therapy, and therapies involving nutraceuticals, plant or animal extracts, fungal extracts, or other natural products.

Pharmaceutical compositions of the present technology can be provided as a single type of composition in any suitable dosage form, or they can be provided as a pharmaceutical kit or pack that includes two or more separate and distinct pharmaceutical compositions, each including nanoparticles containing a different active agent, a different dose of an active agent, or different formulations on one or more active agents. For example, a preferred pharmaceutical kit includes (i) a plurality of first nanoparticles comprising an inhibitor of extracellular vesicle release, and (ii) a second pharmaceutical composition containing a plurality of second nanoparticles comprising an inhibitor of tunneling nanotube formation. The two or more different pharmaceutical compositions can be administered in different amounts, different ratios, or according to different routes or schedules of administration, so as to achieve a desired therapeutic or pharmacokinetic effect.

The pharmaceutical compositions include one or more pharmaceutically acceptable excipients. The pharmaceutical excipients or carriers can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water, saline solutions and aqueous dextrose and glycerol solutions can also be employed as liquid excipients. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Where necessary, the compounds and compositions can also include a solubilizing agent.

The compositions of the present technology can be present in various formulations. Any compound and composition (and/or additional agents) described herein can take the form of solutions, suspensions, emulsion, drops, tablets, pills, pellets, capsules, capsules containing liquids, powders, sustained-release formulations, or any other form suitable for use. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

The compositions can be presented in unit dosage forms and may be prepared by any of the methods well known in the art of pharmacy. In some embodiments, the composition is administered orally. In some embodiments, the administration is by absorption through epithelial or mucocutaneous linings. Compositions for oral delivery can be in the form of tablets, lozenges, aqueous or oily suspensions, granules, powders, emulsions, capsules, syrups, or elixirs, for example. In some embodiments, the compositions are in the form of a capsule, tablet, patch, or lozenge.

The present technology offers several advantages and improvements over previous anti-tumor therapies. These include: simultaneous targeting of different lines of intercellular communication in the solid tumor microenvironment by inhibiting the presence and action of tunneling nanotubes and extracellular vesicles; inhibition of mitochondrial transfer within the tumor microenvironment (a known survival mechanism of cancer cells); inhibiting both multidrug resistance and metastasis; and trapping and improving the effectiveness of low dose anticancer drugs within the tumor microenvironment by inhibiting drug transfer, inhibiting transfer of drug resistance factors, inhibiting mitochondrial transfer, inhibiting cancer cell invasion of other tissues, and inhibiting pre-metastatic niche formation.

The studies presented here have demonstrated that MDR cells have more newly forming TNTs (requiring mitochondria) or are more actively exchanging mitochondria via TNTs, or a combination of both possibilities. The inventors have also demonstrated that co-cultures of a 50:50 ratio of DS and MDR cells results in a high number of TNT formation that more closely resembles MDR TNT formation rates and the co-incubation results in a similar pattern of increased total ATP capacity. Continual mitochondrial exchange is a strategy for maintaining cell survival within the TME; cancer cell mitochondria are at risk of oxidative stress damage, dysfunction due to mitochondrial DNA damage, and alterations in mitophagy and autophagy to maintain survival. Acquiring different mitochondria from neighboring cells is an instant strategy for diluting the effect of mitochondrial damage or dysfunction within a cell. The exchange of mitochondria via TNTs is believed to promote MDR cell survival.

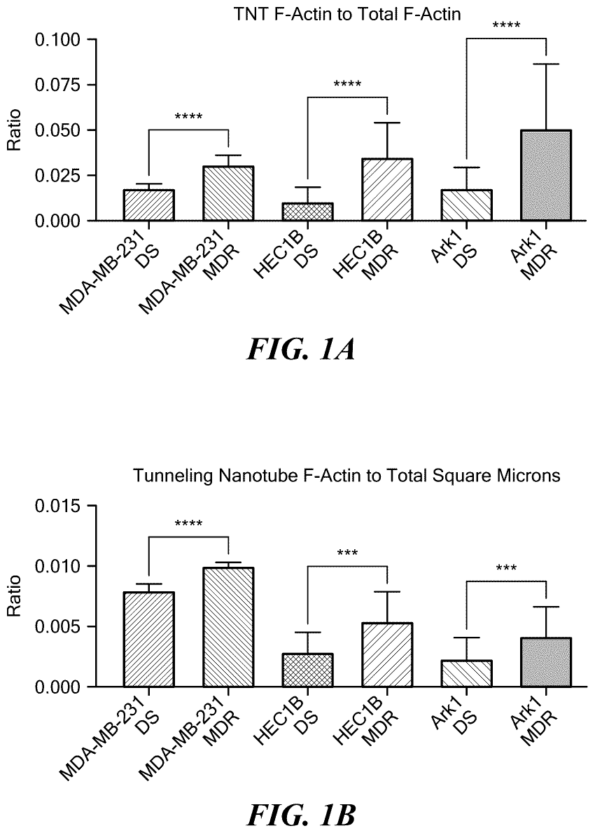

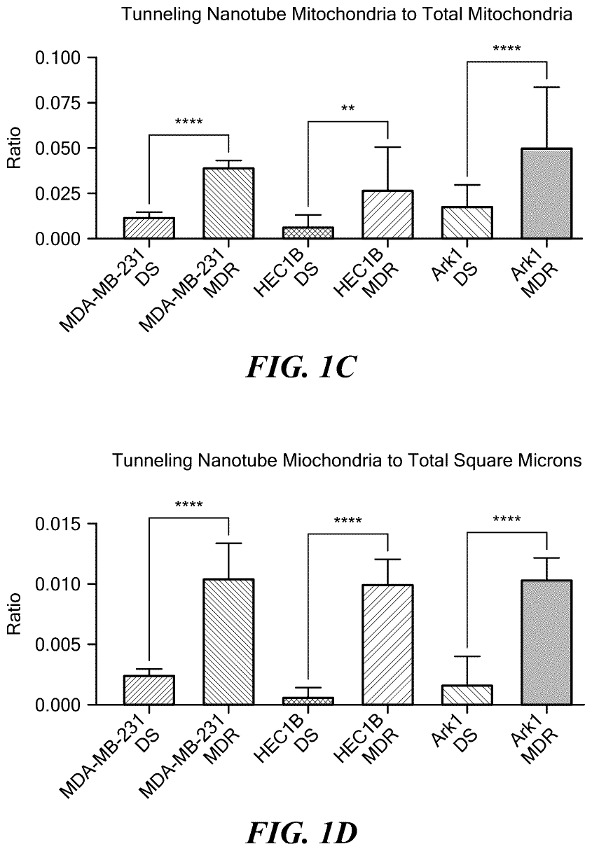

The role of EVs in cancer cell communication, MDR, and metastasis has been investigated^{11-14,29}, but the role of TNTs is less well investigated. MDR is a driver for metastasis through the promotion of epithelial to mesenchymal transition (EMT), a key event required for motility^{10,29-41}. The studies presented here of TNTs in drug sensitive (DS; normoxic) and MDR (hypoxic) cancer cells have demonstrated that MDR cancer cells have more and/or larger TNTs compared to DS cells (FIGS. 1A-1D).

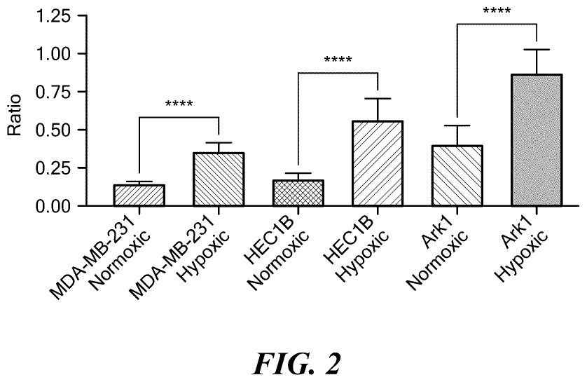

The higher ratio of TNT F-actin to total cellular F-actin (FIG. 1A) and per μm² (FIG. 1B) indicate that the MDR cells have a greater number and/or larger TNTs compared to DS cells. The higher ratio of TNT mitochondria to total cellular mitochondria (FIG. 1C) and per μm² (FIG. 1D), and higher amount of TNT mitochondria to TNT F-actin (FIG. 2) indicates that the TNTs in MDR cells are either more actively transporting and exchanging mitochondria and/or there are more newly synthesized TNTs which requires the presence of mitochondria. By cutting off communication through TNTs, these intercellular highways are prevented from spreading the MDR phenotype within the TME. The present use of nanoparticles is advantageous for treating cancer due to the enhanced permeability and retention (EPR) effect. The leaky, disorganized, and continually remodeled vasculature of a solid tumor and metastatic sites mean that the vascular permeability is higher than normal tissue, allowing a preferential accumulation of nanoparticles relative to normal tissue. As tumors have poor lymphatic drainage, there is increased retention and residence time of accumulated nanoparticles.

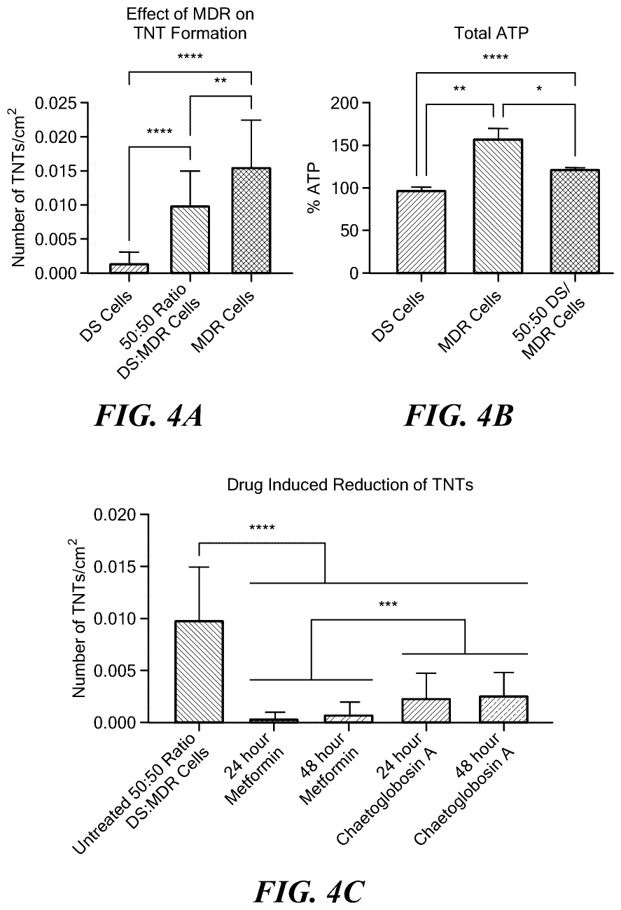

Table 1 summarizes the ex-vivo demonstration of the effectiveness of the present nanoparticle compositions. The nanoparticles inhibited TNT formation and prevented EV release. The nanoparticles combined with a half dose of anticancer drug (such as carboplatin or lenvatinib) is expected to be as effective as the full dose of drug but with a greater phenotype reversal of MDR and with lower toxicity. FIGS. 4A-4C, 5A-5B, and 6A-6C demonstrate the ability of the present nanoparticles to inhibit TNT formation and EV release, and the effect on EV release is believed to be specific to MDR cells, which aids in treatment specificity and decreases off-target toxicity.

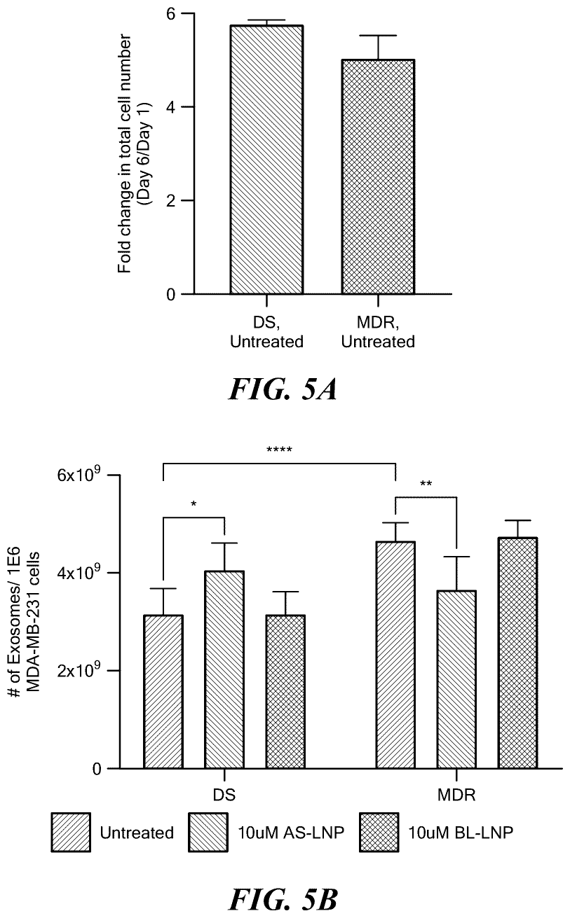

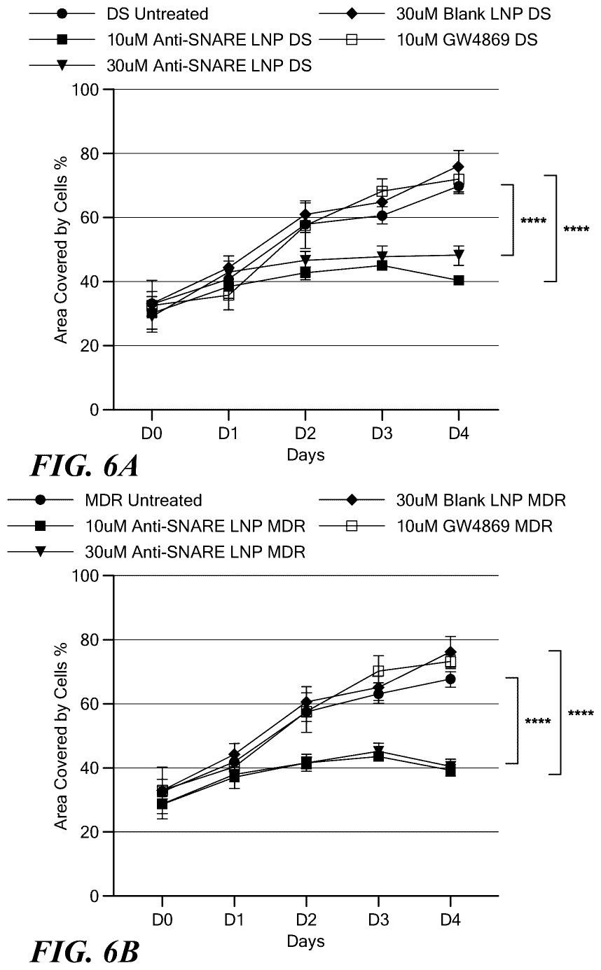

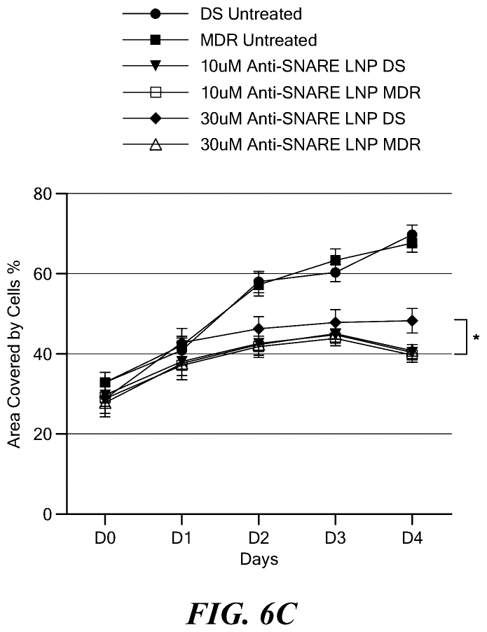

FIGS. 4A-4C demonstrate that MDR cells have more TNTs, and this property is transferred to DS cells in 50/50 co-cultures; this transfer of MDR is inhibited by the present nanoparticles. Similarly FIGS. 5A-5B show that MDR phenotype increases EV release, and the nanoparticles were able to inhibit EV release in MDR cells. Likewise, the nanoparticles inhibited MDR-driven migration (FIGS. 6A-6C). By severing the critical lines of communication in the TME, the present nanoparticles have the potential to transform the treatment of cancers by halting the progression of MDR and increasing the efficacy of anticancer drug therapy.

### Examples

### Example 1. Preparation of Cationic Liposomes

A lipid film was prepared using a cationic lipid, DOTAP (1,2-dioleoyl-3-trimethylammonium-propane chloride salt), together with cholesterol as membrane stabilizer and DPPC (1,2-dipalmitoyl-sn-7 glycero-3-phosphocholine) as a neutral lipid, in a 5:3:5 molar ratio. Two mL of chloroform solution containing the lipids was evaporated using a 10 mL round bottom flask attached to a rotary evaporator. Following chloroform evaporation, the film was resuspended in 1 ml of solution containing 1 mg of anti-SNARE peptide (SEQ ID NO:1) in deionized water, followed by 5 cycles of freezing in liquid nitrogen and heating in a 42° C. water bath. The liposomal preparation was then probe-sonicated for 5 min on ice. The mixture was then centrifuged in an ultracentrifuge at 20,000 RPM for 15 min, using AMICON (10K) centrifugal filters at 4° C. to separate the peptide-encapsulated liposomes from unencapsulated peptide.

### Example 2. Preparation of Biodegradable Polymeric Nanoparticles

Polymeric nanoparticles consisting of 50:50 poly(d,l-lactide-co-glycolide) (PLGA) 7-20k MW and a poly(ethylene glycol) (PEG) (2k MW)-PLGA (10k MW) construct in a 7:3 ratio were synthesized using nanoprecipitation. CA (0.25 mg), PLGA, and PLGA-PEG were dissolved in 2 ml acetone (20 mg total) and allowed to warm in a 37° C. water bath for 10 minutes prior to dropwise addition to 20 ml deionized water under magnetic stirring, following overnight stirring and solvent evaporation, the nanoparticles were pelleted by centrifugation at 10,000g for 30 minutes, and then resuspended in deionized water.

### Example 3. Characterization of Tunnelling Nanotubes in Cancer Cells

FIGS. 1A-1D show characterization of tunnelling nanotubes (TNTs) in drug sensitive and multidrug resistant triple negative breast cancer cells (MDA-MB-231), endometrial cancer cells (HEC1B), and uterine serous carcinoma cells (USC; Ark1). F-Actin and mitochondria were fluorescently labeled and quantified using 3D fluorescence analysis software. FIGS. 1A and 1B show that MDR cells have more and/or larger TNTs than drug sensitive cells as determined by the ratio of TNT F-actin to total cellular F-actin and the TNT F-actin to total area. FIGS. 1C and 1D indicate that TNTs in MDR cells are more active than in drug sensitive cells and/or are newly synthesized, with more mitochondria either supplying energy for synthesis, being actively transported across TNTs, or both. Although significance is only shown within each cell line, the drug sensitive USC cells have more mitochondria that the other drug sensitive lines, and this further increases with MDR. Data are averages of 25 separate 35 mm dishes, plated at 20,000 cells per dish and grown for 5 days. *Welch's t-test.

FIG. 2 shows that TNTs in MDR cells are more actively transporting mitochondria, or there are more newly synthesized TNTs, or both phenomena are occurring simultaneously. F-Actin and mitochondria were fluorescently labeled and quantified using 3D fluorescence analysis software. The increased ratio of TNT mitochondria to TNT F-actin in MDR cells indicates that these TNTs are more actively transporting mitochondria across established TNTs, or that there are more newly synthesized TNTs as mitochondria are required for TNT synthesis, or that both of these conditions are simultaneously occurring in MDR cells. Data are averages of 25 separate 35 mm dishes, plated at 20.000 cells per dish and grown for 5 days. *Welch's t-test.

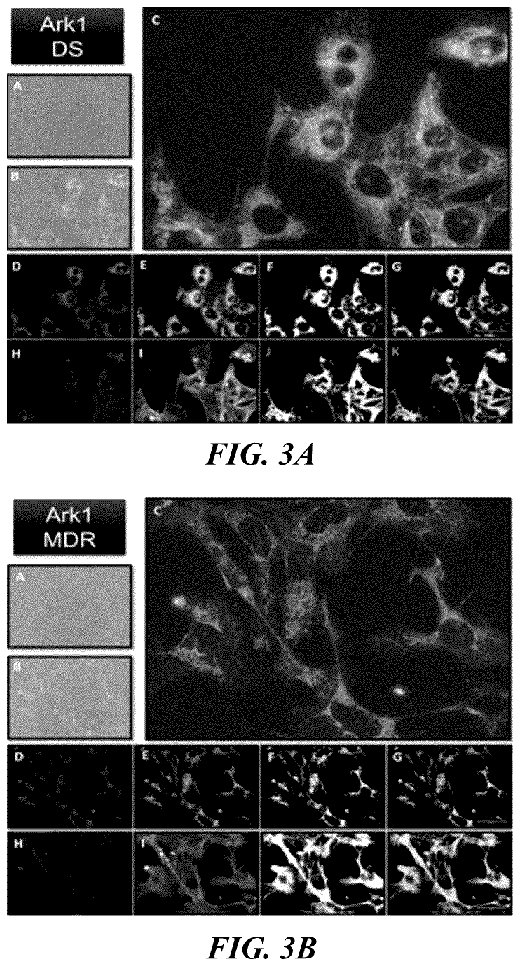

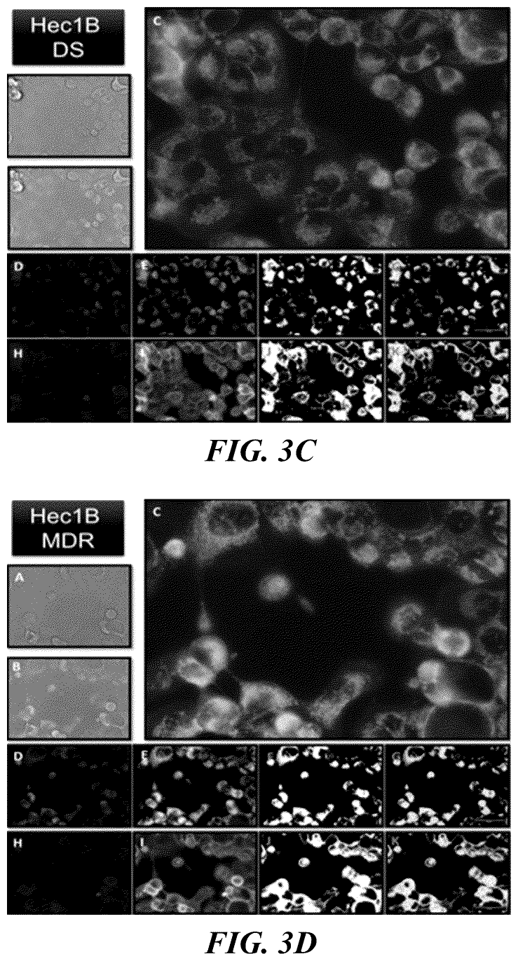

FIGS. 3A-3D show microscopy of untreated drug sensitive and MDR cells. Cells were seeded at 10,000 cells per 35 mm IBIDI glass bottom dish and grown under normoxic (drug sensitive) or hypoxic (MDR: 0.5% O₂) for 5 days. Cells were stained with 62.5 nM SiR-Actin dye (red: F-actin) for 60 min and with 62.5 nM MITOTRACKER Green FM for 15 min. Fluorescence microscopy was performed using the 60X objective of a Keyence BZ-X710 microscope. Images were processed using an adaption of mitochondrial network analysis (MiNA) software for FiJi (NIH) to quantify the 3D fluorescent imprint of F-actin and mitochondria in the entire field of view and in manually identified TNTs. For each figure subpart, panel (A) is brightfield, panel (B) is the microscope overlay of all channels, panel C is the merged overlay of all channels after enhancing local contrast, panels D and H are the mitochondrial green channel and F-actin red channel respectively, panels E and I are the binary conversions of D and H, panels F and J are the 2D analysis of the fluorescent imprint in each channel, and panels G and K are the 3D quantification of fluorescence in each channel. These images demonstrate processing for the entire field of view. The same processing was performed on each individual TNT in the image. This is a representative image of 25 separate fields of view. The data from G and K of the total 25 image sets and the individual TNTs were used to generate the graphs in FIGS. 1A-1D and 2. The scale bars on panels G and K are 64 μm.

FIGS. 4A-4C show that MDR cells have increased TNT formation, and that chaetoglobosin A (CA) decreases TNTs. (FIG. 4A) The number of TNTs in drug sensitive MDA-MB-231 cells, multidrug resistant (MDR) cells, and a 50:50 ratio of co-incubated DS to MDR cells was compared after 5 days of growth. The MDR state increases TNT formation and seems to dominate this increase as the number of TNTs in the 50:50 ratio was more similar to the increase seen in 100% MDR cells. (FIG. 4B) Co-incubation of MDR cells with DS cells also transforms the energetics (total ATP production) as the total ATP capacity of a 50:50 ratio of drug sensitive (DS) to MDR cells more closely resembles the ATP capacity of MDR cells than DS cells. (FIG. 4C) The number of TNTs/cm² were counted in the 50:50 DS/MDR ratio and after treatment with metformin and CA. Metformin was used as a positive control for TNT inhibition. CA significantly reduced the number of TNTs, although metformin was more effective in reducing the number of TNTs. CA is more specific to TNT activity as it is an F-actin inhibitor while the mechanism of action of metformin is multifold. Metformin can be an alternative drug to CA for reducing the number of TNTs. n=25 *Welch's t-test

### Example 4. Characterization of Extracellular Vesicle Release from Cancer Cells

FIGS. 5A-5B show that the MDR state is associated with increased EV release, and that anti-SNARE nanoparticles decreased EVs in MDR cells. (FIG. 5A) The growth rate of drug sensitive and MDR TNBC cells was comparable, allowing direct comparison of the number of EVs per 1 million cells (FIG. 5B). Blank (BL) LNP did not alter the number of EVs. Untreated MDR cells had more EVs than untreated DS cells. although it was not confirmed if this is due to increased EV release, decreased EV uptake, or both it is hypothesized that this is due to a decrease in EV release and this requires SNARE proteins whereas EV uptake is unrelated to SNARE activity. AS-LNP increased EV quantity in DS cells and decreased EV number in MDR cells. The increase in DS cells may be due to a feedback loop/compensation of inhibiting snare proteins. This may lead to higher safety in normal cells and the mechanism of EV increase in DS cells will be further explored. AS-LNP are more effective at decreasing the quantity of EVs in MDR cells which will also be explored in future studies. Notably, this is the anti-SNARE peptide alone and A-COIN is the anti-SNARE peptide combined with CA, which is expected to be more effective as a synergistic combination drug index is expected. n=10. *Welch's t-test

FIGS. 6A-6C show that anti-SNARE nanoparticles decreased cell migration in DS and MDR cells in a wound healing assay. 10 PM and 30 PM AS-LNP reduced cell migration in DS TNBC cells (FIG. 6A) and MDR TNBC cells (FIG. 6B). FIG. 6C shows the results presented in FIGS. 6A and 6B graphed together. Importantly, the exosome inhibitor used as a positive control (GW4869) did not decrease cell migration in DS or MDR cells. These results, and lack of a difference in DS and MDR growth rates, indicate that EVs are not the only cellular process effecting migration, and that EV content is more significant than EV quantity. SNARE proteins mediate EV release but also mediate the fusion of TNTs with recipient cells. The data suggest that anti-SNARE nanoparticles NPs have more specific activity in MDR cells, which provide a safety and efficacy advantage. n=10. *Welch's t-test.

TABLE 1
Nanoparticle Characterization
A. Chaetoglobosin A Loaded Polymeric Nanoparticles

| | | | | Acetone: |
|---|---|---|---|---|
| | PLGA | PEG 2000 | CA | Water |
| Blank | 16 mg | 4 mg | — | 2 ml: 20 ml |
| Nanoparticles | | | | 2 ml: 20 ml |
| CA | 15.975 mg | 4 mg | 0.025 mg | |
| Nanoparticles | | | | |
| | | Hydrodynamic | Zeta | |
| | | Diameter | Potential | Encapsulation |
| | | (nm) | (mV) | Efficiency (%) |
| Blank Nanoparticles | | 107.7 ± 32.43 | 83.6 ± 6.08 | — |
| CA Nanoparticles | | 150.2 ± 48.64 | 4.07 ± 4.03 | 68.9 ± 6.27 |

B. Anti-SNARE Peptide Loaded Liposomes

| | DPPC | Cholesterol | 18:0 PEG 2000 | Chloroform |
|---|---|---|---|---|
| Volume (μL) | 234.0 | 34.0 | 56.0 | 676.0 |
| Weight (mg) | 5.85 | 0.8506 | 1.40 | — |
| Molar Ratio | 7.2 | 2.2 | 0.5 | — |

| | | | Encapsulation |
|---|---|---|---|
| | | Zeta Potential | (mV) |
| | Hydrodynamic | Diameter (nm) | Efficiency (%) |
| Blank Liposomes | 184.8 ± 5.679 | −22.5 ± 6.59 | N/A |
| Anti-SNARE Liposomes | 189.9 ± 8.958 | −25.2 ± 4.43 | 97.16 ± 2.13 |

As used herein, “consisting essentially of” allows the inclusion of materials or steps that do not materially affect the basic and novel characteristics of the claim. Any recitation herein of the term “comprising”, particularly in a listing of components of a composition or elements of a device, constitutes inclusion of alternative embodiments in which “comprising” is replaced with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

### REFERENCES

1 American Cancer Society, I. Key Statistics for Endometrial Cancer, <www.cancer.org/cancer/endometrial-cancer/about/key-statistics.html> (2021).
2 Makker, V. et al. New therapies for advanced, recurrent, and metastatic endometrial cancers. Gynecologic Oncology Research and Practice 4, 19, doi: 10.1186/s40661-017-0056-7 (2017). PMID: 29214032
3 Morice, P., Leary, A., Creutzberg, C., Abu-Rustum, N. & Darai, E. Endometrial cancer. Lancet 387, 1094-1108, doi: 10.1016/s0140-6736 (15) 00130-0 (2016). PMID: 26354523
4 NCI. <www.cancer.gov/about-cancer/understanding/statistics> (5
5. NCI. Identifying Novel Drug Combinations to Overcome Treatment Resistance, <www.cancer.gov/about-cancer/treatment/research/drug-combo-resistance> (2016).
6 Institute, N. C. <www.cancer.gov/research/annual-plan/scientific-topics/what-is-drug-resistance-infographic> (2020).
7 Network, N. C. C. (2021).
8 Desir, S. et al. Chemotherapy-Induced Tunneling Nanotubes Mediate Intercellular Drug Efflux in Pancreatic Cancer. Scientific Reports 8, 9484, doi: 10.1038/s41598-018-27649-x (2018). PMID: 29930346
9 Hanna, S. J. et al. Tunneling nanotubes, a novel mode of tumor cell-macrophage communication in tumor cell invasion. J Cell Sci 132, doi: 10.1242/jcs.223321 (2019). PMID: 30659112
10 Bouvy, C., Wannez, A., Laloy, J., Chatelain, C. & Dogné, J.-M. Transfer of multidrug resistance among acute myeloid leukemia cells via extracellular vesicles and their microRNA cargo. Leukemia Research 62, 70-76, doi: doi.org/10.1016/j.leukres.2017.09.014 (2017). PMID:
11 Milane, L., Singh, A., Mattheolabakis, G., Suresh, M. & Amiji, M. M. Exosome mediated communication within the tumor microenvironment. J Control Release 219, 278-294, doi: 10.1016/j.jconrel.2015.06.029 (2015). PMID: 26143224
12 Ogawa, K. et al. Prometastatic secretome trafficking via exosomes initiates pancreatic cancer pulmonary metastasis. Cancer Letters 481, 63-75, doi: doi.org/10.1016/j.canlet.2020.02.039 (2020). PMID: 32145343
13 Bandari, S. K. et al. Chemotherapy induces secretion of exosomes loaded with heparanase that degrades extracellular matrix and impacts tumor and host cell behavior. Matrix Biology 65, 104-118, doi: doi.org/10.1016/j.matbio.2017.09.001 (2018). PMID: 28888912
14 Feng, T. et al. High throughput sequencing identifies breast cancer-secreted exosomal LncRNAs initiating pulmonary pre-metastatic niche formation. Gene 710, 258-264, doi: doi.org/10.1016/j.gene.2019.06.004 (2019). PMID: 31176731
15 Abounit, S. & Zurzolo, C. Wiring through tunneling nanotubes-from electrical signals to organelle transfer. Journal of Cell Science 125, 1089-1098, doi: 10.1242/jcs.083279 (2012). PMID: 22399801
16 Onfelt, B. et al. Structurally distinct membrane nanotubes between human macrophages support long-distance vesicular traffic or surfing of bacteria. Journal of immunology (Baltimore, Md.: 1950) 177, 8476-8483, doi: 10.4049/jimmunol. 177.12.8476 (2006). PMID: 17142745
17 Wang, S. et al. Investigating tunneling nanotubes in ovarian cancer based on two-photon excitation FLIM-FRET. Biomed Opt Express 12, 1962-1973, doi: 10.1364/boe.418778 (2021). PMID: 33996210
18 Borcherding, N. & Brestoff, J. R. The power and potential of mitochondria transfer. Nature 623, 283-291, doi: 10.1038/s41586-023-06537-z (2023). PMID: 37938702
19 Lou, E. A Ticket to Ride: The Implications of Direct Intercellular Communication via Tunneling Nanotubes in Peritoneal and Other Invasive Malignancies. Frontiers in Oncology 10, doi: 10.3389/fonc.2020.559548 (2020). PMID: 33324545
20 Pasquier, J. et al. Preferential transfer of mitochondria from endothelial to cancer cells through tunneling nanotubes modulates chemoresistance. J Transl Med 11, 94, doi: 10.1186/1479-5876-11-94 (2013). PMID: 23574623
21 Goliwas, K. F. et al. Mitochondrial transfer from cancer-associated fibroblasts increases migration in aggressive breast cancer. Journal of Cell Science 136, doi: 10.1242/jcs.260419 (2023). PMID: 37358264
22 Saha, T. et al. Intercellular nanotubes mediate mitochondrial trafficking between cancer and immune cells. Nature nanotechnology 17, 98-106, doi: 10.1038/s41565-021-01000-4 (2022). PMID: 34795441
23 Roehlecke, C. & Schmidt, M. H. H. Tunneling Nanotubes and Tumor Microtubes in Cancer. Cancers (Basel) 12, doi: 10.3390/cancers12040857 (2020). PMID: 32244839
24 Valdebenito, S. et al. Tunneling nanotubes, TNT, communicate glioblastoma with surrounding non-tumor astrocytes to adapt them to hypoxic and metabolic tumor conditions. Scientific Reports 11, 14556, doi: 10.1038/s41598-021-93775-8 (2021). PMID: 34267246
25 Wang, X. & Gerdes, H. H. Transfer of mitochondria via tunneling nanotubes rescues apoptotic PC12 cells. Cell Death & Differentiation 22, 1181-1191, doi: 10.1038/cdd.2014.211 (2015). PMID: 25571977
26 Saha, T. et al. Intercellular nanotubes mediate mitochondrial trafficking between cancer and immune cells. Nature nanotechnology 17, 98-106, doi: 10.1038/s41565-021-01000-4 (2022). PMID: 34795441
27 Thayanithy, V., Dickson, E. L., Steer, C., Subramanian, S. & Lou, E. Tumor-stromal cross talk: direct cell-to-cell transfer of oncogenic microRNAs via tunneling nanotubes. Transl Res 164, 359-365, doi: 10.1016/j.trsl.2014.05.011 (2014). PMID: 24929208
28 Fader, C. M., Savina, A., Sanchez, D. & Colombo, M. I. Exosome secretion and red cell maturation: Exploring molecular components involved in the docking and fusion of multivesicular bodies in K562 cells. Blood Cells, Molecules, and Diseases 35, 153-157, doi: doi.org/10.1016/j.bcmd.2005.07.002 (2005). PMID: 16099697
29 Balaji, S., Kim, U., Muthukkaruppan, V. & Vanniarajan, A. Emerging role of tumor microenvironment derived exosomes in therapeutic resistance and metastasis through epithelial-to-mesenchymal transition. Life Sciences 280, 119750, doi: doi.org/10.1016/j.lfs.2021.119750 (2021). PMID: 34171378
30 Comerford, K. M. et al. Hypoxia-inducible Factor-1-dependent Regulation of the Multidrug Resistance Gene. Cancer Research 62, 3387 (2002). PMID: 12067980
31 Giddings, E. L. et al. Mitochondrial ATP fuels ABC transporter-mediated drug efflux in cancer chemoresistance. Nature Communications 12, 2804, doi: 10.1038/s41467-021-23071-6 (2021).
32 Gottesman, M. M., Fojo, T. & Bates, S. E. Multidrug resistance in cancer: role of ATP-dependent transporters. Nature Reviews Cancer 2, 48, doi: 10.1038/nrc706 (2002). PMID: 11902585
33 Hekmatshoar, Y., Nakhle, J., Galloni, M. & Vignais, M.-L. The role of metabolism and tunneling nanotube-mediated intercellular mitochondria exchange in cancer drug resistance. Biochemical Journal 475, 2305-2328, doi: 10.1042/bcj20170712 (2018). PMID: 30064989
34 Housman, G. et al. Drug Resistance in Cancer: An Overview. Cancers 6, 1769-1792, doi: 10.3390/cancers6031769 (2014). PMID: 25198391
35 Juan-Carlos, P.-D. M., Perla-Lidia, P.-P., Stephanie-Talia, M.-M., Mónica-Griselda, A.-M. & Luz-María, T.-E. ABC transporter superfamily. An updated overview, relevance in cancer multidrug resistance and perspectives with personalized medicine. Molecular Biology Reports 48, 1883-1901, doi: 10.1007/s11033-021-06155-w (2021). PMID: 33616835
36 Milane, L., Duan, Z. & Amiji, M. Role of hypoxia and glycolysis in the development of multi-drug resistance in human tumor cells and the establishment of an orthotopic multi-drug resistant tumor model in nude mice using hypoxic pre-conditioning. Cancer Cell Int 11, 3, doi: 10.1186/1475-2867 Nov. 3 (2011). PMID: 21320311
37 Milane, L., Ganesh, S., Shah, S., Duan, Z. F. & Amiji, M. Multi-modal strategies for overcoming tumor drug resistance: hypoxia, the Warburg effect, stem cells, and multifunctional nanotechnology. J Control Release 155, 237-247, doi: 10.1016/j.jconrel.2011.03.032 (2011). PMID: 21497176
38 Sodani, K., Patel, A., Kathawala, R. J. & Chen, Z.-S. Multidrug resistance associated proteins in multidrug resistance. Chin J Cancer 31, 58-72, doi: 10.5732/cjc.011.10329 (2012). PMID: 22098952
39 Yousafzai, N. A. et al. Exosome mediated multidrug resistance in cancer. American journal of cancer research 8, 2210-2226 (2018). PMID: 30555739
40 Fares, J., Fares, M. Y., Khachfe, H. H., Salhab, H. A. & Fares, Y. Molecular principles of metastasis: a hallmark of cancer revisited. Signal Transduction and Targeted Therapy 5, 28, doi: 10.1038/s41392-020-0134-x (2020). PMID: 32296047
41 Makker, A. & Goel, M. M. Tumor progression, metastasis, and modulators of epithelial-mesenchymal transition in endometrioid endometrial carcinoma: an update. Endocrine-Related Cancer 23, R85-R111, doi: 10.1530/ERC-15-0218 (2016). PMID: 26538531
42 Boekweg, H., Guise, A. J., Plowey, E. D., Kelly, R. T. & Payne, S. H. Calculating Sample Size Requirements for Temporal Dynamics in Single-Cell Proteomics. Molecular & Cellular Proteomics 20, 100085, doi: doi.org/10.1016/j.mcpro.2021.100085 (2021). PMID: 33915259
43 Dell, R. B., Holleran, S. & Ramakrishnan, R. Sample size determination. ILAR J 43, 207-213, doi: 10.1093/ilar.43.4.207 (2002). PMID: 12391396
44 Bagiella, E. & Chang, H. Power analysis and sample size calculation. Journal of Molecular and Cellular Cardiology 133, 214-216, doi: doi.org/10.1016/j.yjmcc.2019.01.006 (2019). PMID: 30639699
45 Shiku, H. et al. Metabolic and enzymatic activities of individual cells, spheroids and embryos as a function of the sample size. Sensors and Actuators B: Chemical 108, 597-602, doi: doi.org/10.1016/j.snb.2004.12.030 (2005).
46 Ellis, P. The Essential Guide to Effect Sizes Statistical Power, Meta-Analysis, and the Interpretation of Research Results. (Cambridge University Press, 2010).
47 Blanes-Mira, C. et al. Identification of SNARE complex modulators that inhibit exocytosis from an alpha-helix-constrained combinatorial library. The Biochemical journal 375, 159-166, doi: 10.1042/BJ20030509 (2003). PMID: 12852787
48 Pawar, G. N., Parayath, N. N., Nocera, A. L., Bleier, B. S. & Amiji, M. M. Direct CNS delivery of proteins using thermosensitive liposome-in-gel carrier by heterotopic mucosal engrafting. PLOS ONE 13, e0208122, doi: 10.1371/journal.pone.0208122 (2018). PMID: 30517163
49 Franco, A. et al. Correcting mitochondrial fusion by manipulating mitofusin conformations. Nature 540, 74-79, doi: 10.1038/nature20156 (2016). PMID: 27775718
50 Breen, L., Murphy, L., Keenan, J. & Clynes, M. Development of taxane resistance in a panel of human lung cancer cell lines. Toxicol In Vitro 22, 1234-1241, doi: 10.1016/j.tiv.2008.04.005 (2008). PMID: 18514476
51 McDermott, M. et al. In vitro Development of Chemotherapy and Targeted Therapy Drug-Resistant Cancer Cell Lines: A Practical Guide with Case Studies. Front Oncol 4, 40, doi: 10.3389/fonc.2014.00040 (2014). PMID: 24639951
52 Valente, A. J., Maddalena, L. A., Robb, E. L., Moradi, F. & Stuart, J. A. A simple ImageJ macro tool for analyzing mitochondrial network morphology in mammalian cell culture. Acta Histochem 119, 315-326, doi: 10.1016/j.acthis.2017.03.001 (2017). PMID: 28314612
53 Jonkman, J. E. N. et al. An introduction to the wound healing assay using live-cell microscopy. Cell Adh Migr 8, 440-451, doi: 10.4161/cam.36224 (2014). PMID: 25482647
54 Pijuan, J. et al. In vitro Cell Migration, Invasion, and Adhesion Assays: From Cell Imaging to Data Analysis. Front Cell Dev Biol 7, doi: 10.3389/fcell.2019.00107 (2019). PMID: 31259172
55 Luther, E., Mendes, L. P., Pan, J., Costa, D. F. & Torchilin, V. P. Applications of label-free, quantitative phase holographic imaging cytometry to the development of multi-specific nanoscale pharmaceutical formulations. Cytometry A 91, 412-423, doi: 10.1002/cyto.a.23102 (2017). PMID: 28371272
56 Ariffin, N. S. The CellProfiler pipeline analysis of cell migration. Acta Histochem 125, 152074, doi: 10.1016/j.acthis.2023.152074 (2023). PMID: 37437311
57 Chou, T.-C. Drug Combination Studies and Their Synergy Quantification Using the Chou-Talalay Method. Cancer Research 70, 440-446, doi: 10.1158/0008-5472.can-09-1947 (2010). PMID: 20068163
58 Eavarone, D. A. et al. Humanized anti-Sialyl-Tn antibodies for the treatment of ovarian carcinoma. PLOS One 13, e0201314, doi: 10.1371/journal.pone.0201314 (2018). PMID: 30052649
59 Fedorko, A. M. et al. An immune competent orthotopic model of endometrial cancer with metastasis. Heliyon 6, e04075, doi: 10.1016/j.heliyon.2020.e04075 (2020). PMID: 32490257
60 Li, P. et al. Lung mesenchymal cells elicit lipid storage in neutrophils that fuel breast cancer lung metastasis. Nat Immunol 21, 1444-1455, doi: 10.1038/s41590-020-0783-5 (2020). PMID: 32958928
61 Li, P. et al. Dual roles of neutrophils in metastatic colonization are governed by the host NK cell status. Nat Commun 11, 4387, doi: 10.1038/s41467-020-18125-0 (2020). PMID: 32873795
62 Zheng, H. et al. Therapeutic Antibody Targeting Tumor- and Osteoblastic Niche-Derived Jagged1 Sensitizes Bone Metastasis to Chemotherapy. Cancer Cell 32, 731-747 e736, doi: 10.1016/j.ccell.2017.11.002 (2017). PMID: 29232552
63 Gong, Z. et al. Lung fibroblasts facilitate pre-metastatic niche formation by remodeling the local immune microenvironment. Immunity, doi: 10.1016/j.immuni.2022.07.001 (2022). PMID:
64 Zhang, P., Sun, Y. & Ma, L. ZEB1: at the crossroads of epithelial-mesenchymal transition, metastasis and therapy resistance. Cell cycle (Georgetown, Tex.) 14, 481-487, doi: 10.1080/15384101.2015.1006048 (2015). PMID: 25607528
65 Yao, D., Dai, C. & Peng, S. Mechanism of the Mesenchymal-Epithelial Transition and Its Relationship with Metastatic Tumor Formation. Molecular Cancer Research 9, 1608-1620, doi: 10.1158/1541-7786.mcr-10-0568 (2011). PMID: 21840933
66 Vasaikar, S. V. et al. EMTome: a resource for pan-cancer analysis of epithelial-mesenchymal transition genes and signatures. British Journal of Cancer 124, 259-269, doi: 10.1038/s41416-020-01178-9 (2021). PMID: 33299129
67 Pan, G., Liu, Y., Shang, L., Zhou, F. & Yang, S. EMT-associated microRNAs and their roles in cancer stemness and drug resistance. Cancer Communications 41, 199-217, doi: doi.org/10.1002/cac2.12138 (2021). PMID: 33506604
68 Ceppi, P. & Peter, M. E. MicroRNAs regulate both epithelial-to-mesenchymal transition and cancer stem cells. Oncogene 33, 269-278, doi: 10.1038/onc.2013.55 (2014). PMID: 23455327
69 Au-Philp, L. et al. An Orthotopic Endometrial Cancer Model with Retroperitoneal Lymphadenopathy Made From In Vivo Propagated and Cultured VX2 Cells. JoVE, e59340, doi: doi: 10.3791/59340 (2019). PMID: 31566617
70 Philp, L. et al. Use of Porphysomes to detect primary tumour, lymph node metastases, intra-abdominal metastases and as a tool for image-guided lymphadenectomy: proof of concept in endometrial cancer. Theranostics 9, 2727-2738, doi: 10.7150/thno.31225 (2019). PMID: 31131064
71 Seebacher, N. A., Krchniakova, M., Stacy, A. E., Skoda, J. & Jansson, P. J. Tumour Microenvironment Stress Promotes the Development of Drug Resistance. Antioxidants (Basel) 10, doi: 10.3390/antiox10111801 (2021). PMID: 34829672
72 Milane, L., Duan, Z. & Amiji, M. Therapeutic efficacy and safety of paclitaxel/lonidamine loaded EGFR-targeted nanoparticles for the treatment of multi-drug resistant cancer. PLOS One 6, e24075, doi: 10.1371/journal.pone.0024075 (2011). PMID: 21931642
73 Milane, L., Duan, Z. & Amiji, M. Role of hypoxia and glycolysis in the development of multi-drug resistance in human tumor cells and the establishment of an orthotopic multi-drug resistant tumor model in nude mice using hypoxic pre-conditioning. Cancer Cell Int 11, 3, doi: 10.1186/1475-2867 Nov. 3 (2011). PMID: 21320311
74 Milane, L., Duan, Z. & Amiji, M. Development of EGFR-targeted polymer blend nanocarriers for combination paclitaxel/lonidamine delivery to treat multi-drug resistance in human breast and ovarian tumor cells. Mol Pharm 8, 185-203, doi: 10.1021/mp1002653 (2011). PMID: 20942457
75 Milane, L., Duan, Z. F. & Amiji, M. Pharmacokinetics and biodistribution of lonidamine/paclitaxel loaded, EGFR-targeted nanoparticles in an orthotopic animal model of multi-drug resistant breast cancer. Nanomedicine 7, 435-444, doi: 10.1016/j.nano.2010.12.009 (2011). PMID: 21220050
76 Famta, P. et al. Tumor-promoting aftermath post-chemotherapy: A focus on breast cancer. Life Sciences 310, 121125, doi: doi.org/10.1016/j.lfs.2022.121125 (2022). PMID: 36306868
77 Wei, Z. et al. Exosomes derived from MDR cells induce cetuximab resistance in CRC via PI3K/AKT signaling-mediated Sox2 and PD-L1 expression. Exp Ther Med 25, 86, doi: 10.3892/etm.2023.11785 (2023). PMID: 36741914
78 Emran, T. B. et al. Multidrug Resistance in Cancer: Understanding Molecular Mechanisms, Immunoprevention and Therapeutic Approaches. Front Oncol 12, 891652, doi: 10.3389/fonc.2022.891652 (2022). PMID: 35814435
79 Bellio, C. et al. PARP Inhibition Induces Enrichment of DNA Repair-Proficient CD133 and CD117 Positive Ovarian Cancer Stem Cells. Molecular Cancer Research 17, 431-445, doi: 10.1158/1541-7786.Mcr-18-0594 (2019). PMID: 30401718
80 Kulkarni-Datar, K., Orsulic, S., Foster, R. & Rueda, B. R. Ovarian tumor initiating cell populations persist following paclitaxel and carboplatin chemotherapy treatment in vivo. Cancer Letters 339, 237-246, doi: doi.org/10.1016/j.canlet.2013.06.014 (2013). PMID: 23791886
81 Kawahara, T; Itoh, M; Izumikawa, M; Sakata, N; Tsuchida, T; Shin-Ya, K (2013). “New chaetoglobosin derivatives, MBJ-0038, MBJ-0039 and MBJ-0040, isolated from the fungus Chaetomium sp. f24230”. The Journal of Antibiotics. 66 (12): 727-30

1. A pharmaceutical composition for treating a solid tumor in a mammalian subject, the pharmaceutical composition comprising a plurality of nanoparticles comprising an inhibitor of extracellular vesicle release and/or an inhibitor of tunneling nanotube formation.

2. The pharmaceutical composition of claim 1, wherein the inhibitor of extracellular vesicle release is an anti-SNARE compound.

3. The pharmaceutical composition of claim 2, wherein the anti-SNARE compound is a peptide comprising the amino acid sequence SAAEAFAKLYAEAFAKG (SEQ ID NO: 1) or a fragment or variant thereof having at least 70%, at least 80%, at least 90%, at least 95%, at least 98%, or at least 99% identity to SEQ ID NO:1.

4. The pharmaceutical composition of claim 2, wherein the anti-SNARE compound is a peptide comprising the amino acid sequence EEVLQLMRRTSEL (SEQ ID NO:2), NRRLQQTQAQVDEVVDIMRVNVDKVLER (SEQ ID NO:3), or DQKLSELDDRADALQAGASQFETSAAKLKRKYWWKN (SEQ ID NO:4), or a fragment or variant thereof having at least 70%, at least 80%, at least 90%, at least 95%, at least 98%, or at least 99% identity to any of SEQ ID NOS: 2-4.

5. The pharmaceutical composition of claim 3, wherein the anti-SNARE compound is a variant of any of SEQ ID NOS: 1-4 comprising from 1 to 10, from 1 to 5, or from 1 to 3 conservative amino acid substitutions.

6. The pharmaceutical composition of claim 1, wherein the inhibitor of tunneling nanotube formation is an inhibitor of F-actin.

7. The pharmaceutical composition of claim 1, wherein the inhibitor of tunneling nanotube formation is chaetoglobosin A, cytochalasin B, or metformin.

8. The pharmaceutical composition of claim 1, wherein the nanoparticles comprise cationic liposomes or biodegradable polymeric nanoparticles, and wherein the cationic liposomes or polymeric nanoparticles encapsulate the inhibitor of extracellular vesicle release and/or the inhibitor of tunneling nanotube formation.

9. The pharmaceutical composition of claim 8, wherein the nanoparticles are cationic liposomes.

10. The pharmaceutical composition of claim 6, wherein the cationic liposomes comprise 1,2-dioleoyl-3-trimethylammonium-propane (DOTAP) as the cationic lipid and further comprise one or more components selected from the group consisting of a neutral phospholipid, cholesterol, and a hydrophilic polymer such as polyethylene glycol (PEG).

11. The pharmaceutical composition of claim 10, wherein the cationic liposomes have a zeta potential greater than about +30 mV or less than about −30 mV.

12. The pharmaceutical composition of claim 8, wherein the nanoparticles are biodegradable polymeric nanoparticles.

13. The pharmaceutical composition of claim 1, wherein the anti-SNARE compound and the inhibitor of tunneling nanotube formation are co-localized within the same cationic liposomes or within the same biodegradable polymeric nanoparticles.

14. The pharmaceutical composition of claim 1, wherein the anti-SNARE compound and the inhibitor of tunneling nanotube formation are localized within separate cationic liposomes or within separate biodegradable polymeric nanoparticles.

15. A method of treating a solid tumor-forming cancer in a mammalian subject, the method comprising administering the pharmaceutical composition of claim 1 to the mammalian subject.

16. The method of claim 15, wherein release of extracellular vesicles and/or formation, number, or density of tunneling nanotubes are inhibited in a solid tumor of the mammalian subject.

17. The method of claim 15, wherein metastasis of cancer cells out of the tumor and/or development or spread of multidrug resistant cancer cells within the tumor or within the subject are inhibited.

18. The method of claim 15, further comprising administering an anticancer drug to the mammalian subject, prior to, concomitantly with, or after administration of said pharmaceutical composition.

19. The method of claim 18, wherein the anticancer drug is administered at a lower dose than would be administered if said pharmaceutical were not also being administered.

20. A pharmaceutical kit, comprising;
(i) a first pharmaceutical composition comprising a plurality of first nanoparticles comprising an inhibitor of extracellular vesicle release; and
(ii) a second pharmaceutical composition comprising a plurality of second nanoparticles comprising an inhibitor of tunneling nanotube formation.
